(19) United States
(12) Patent Application Publication
(10) Pub. No.: US 2005/0261553 A1
(43) Pub. Date: Nov. 24, 2005

(54) Endoscope, for example a colonoscope, and a cleaning method for use therewith

(76) Inventors: Christopher P. Swain, London (GB); Charles A. Mosse, London (GB); Annette D.A. Fritscher-Ravens, Bruchhausen-Vilsen (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/115,677

(22) Filed: Apr. 27, 2005

(30) Foreign Application Priority Data
Apr. 28, 2004 (GB) UK0409474.4

(51) Int. Cl. A61B 1/015

(52) U.S. Cl. 600/157; 600/156

(57) ABSTRACT

An apparatus and method for cleaning a body cavity, such as a portion of the GI tract, are disclosed. The apparatus can include a colonoscope having viewing optics and a liquid conduit with at least one outlet. The outlet can be positioned to direct a liquid to clean the colon or other cavity in the region being viewed.

This patent application claims priority to and incorporates by reference UK Patent Application 0409474.4 “Colonoscope and a Colon Cleaning Method for Use Therewith” filed Apr. 28, 2004.

This invention relates to medical devices and methods and general, and more particularly to method and devices useful in endoscopy, such as an endoscope for examination of a human or animal cavity.

### BACKGROUND OF THE INVENTION

A practice in carrying out colonoscopy is to give the patients, prior to colonoscopy, an oral preparation which is to be taken to clean the colon. However, some patients do not take the preparation as prescribed or do not respond to it. In particular, patients with bleeding in the lower part of the gastro-intestinal tract, and patients who are already hospitalised, are hard to prepare prior to colonoscopy. As a result, a significant percentage of colonoscopies may have to be postponed to another day because of poor preparation, and in some cases the colonoscopy examination may be incomplete. Also, patients may find that the diarrhoea and cramping abdominal pain, which may follow administration of the oral preparation, may cause more discomfort than the colonoscopy itself.

U.S. Pat. No. 4,682,979 (Girouard) discloses passing into the colon a flexible tube having fluid nozzles at its distal end. U.S. Pat. No. 4,735,501 (Ginsburgh et al), discloses fluid propulsion of borescopes and the application of fluid propulsion to endoscopes.

### SUMMARY OF THE INVENTION

Applicant's have recognized the desirability of providing a method of cleaning a portion of the gastro-intestinal tract (such as the colon) which can be used instead of, or as a supplement to, the use of conventional preparations administered to the patient.

According to one embodiment of the present invention there is provided an endoscope, for example a colonoscope, comprising a main body having a distal end for insertion into a cavity in a human or animal body, for example a colon, and a proximal end; viewing optics having a viewing port at or adjacent the distal end of the main body, whereby to view a region distal of the viewing port; and a liquid conduit having a distal end portion with at least one outlet at or adjacent the distal end of the main body, and a proximal end portion arranged, in use, to receive a supply of liquid, said at least one outlet being arranged, in use, to direct said liquid to clean the cavity in the region being viewed.

In one embodiment, each fluid outlet is positioned forwardly of the viewing port, and can be directed, at least partly, towards the proximal end of the colonoscope main body (i.e. towards the anus of the patient). Directing the fluid in this way has the advantage that it helps to set up a flow of fluid to carry stools broken up by the jets, in the form of a slurry, towards the anus. It is to be understood, however, that the jets could be directed purely laterally, or even forwardly.

The invention also provides a method for cleaning a portion of a body cavity in a patient. In one embodiment, the method can include the steps of positioning a viewing port within a cavity, such as in a portion of the colon; positioning a fluid conduit having at least one outlet viewable by the viewing port; directing liquid from the outlet to clean the cavity; and collecting at least some of the liquid used in cleaning.

In one embodiment, the method can include the steps of positioning a viewing port within the passage; positioning a fluid conduit having at least one outlet viewable by the viewing port; directing liquid from the outlet; and forming a slurry comprising material, such as fecal material, disposed within the cavity.

### BRIEF DESCRIPTION OF THE DRAWINGS:

The invention is described further below with reference to the accompanying drawings, in which:

FIG. 1 shows the distal part of a first embodiment of a colonoscope according to the present invention located in a colon, the anus being understood to be located leftwardly of the colon portion shown;

FIG. 2 is a similar view of a second embodiment;

FIG. 2a shows, in end view, and on a larger scale, a tip member forming part of the embodiment of FIG. 2.

FIG. 3 shows one possible arrangement for collecting the slurry produced by the cleaning method;

FIG. 4 shows an alternative collection arrangement; and

FIG. 4a shows part of one possible form of suction tube used in FIG. 4.

### DETAILED DESCRIPTION

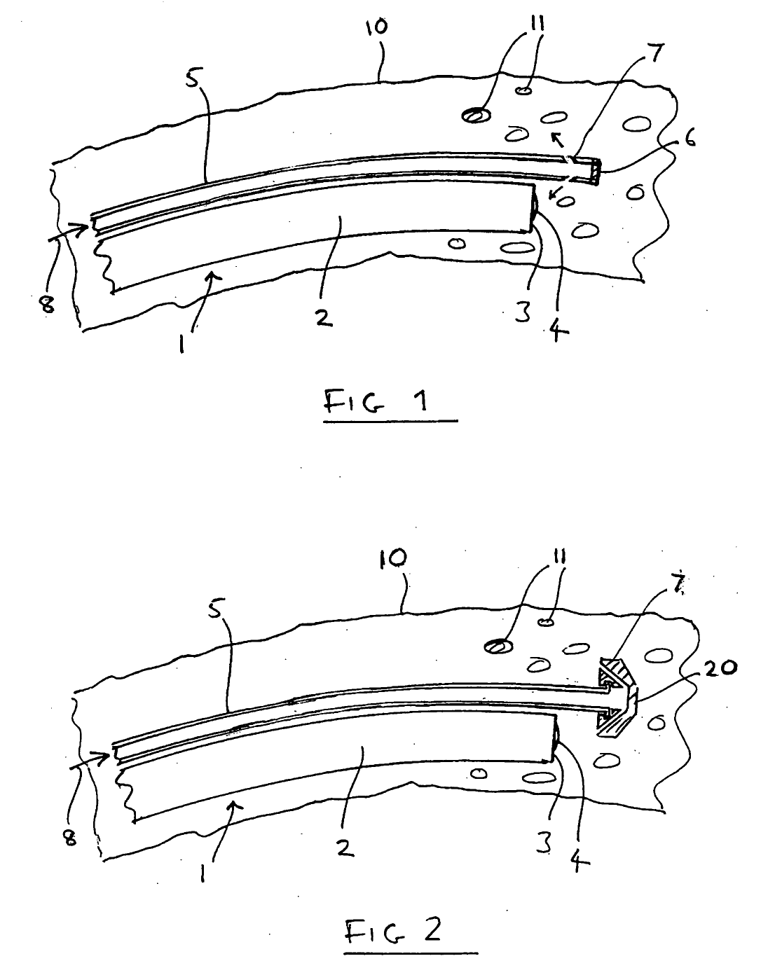

FIG. 1 shows, in part, a colonoscope 1 having a flexible main body 2 with a distal end 3 and a proximal end (not shown). The viewing optics of the colonoscope terminate in a window 4. A conduit 5 for carrying a cleaning liquid runs parallel to the main body 2, and terminates at a closed distal end 6. The distal end 6 can be located forwardly of the distal end 3 of the main body by a short distance of, for example, about 1 cm to about 2 cm. Alternatively, this distance can be greater than or less than the range of 1 to 2 cm. The portion of the conduit 5 forwardly of the distal end 3 is provided with at least one fluid outlet 7. The embodiment of FIG. 1 has four such outlets, arranged at 90° to one another around the distal end, of which two are visible in FIG. 1. However, it is to be understood that there might be only one, two or three outlets, or there might be more than four. Liquid under pressure enters the conduit 5 from its proximal end, with its direction of flow being indicated by arrow 8, and leaves the conduit 5 through the outlets 7. These are preferably angled backwardly, as shown in FIG. 1, so that the direction of fluid flow as it leaves the outlets has a component directed towards the proximal end of the endoscope and thus towards the patient's anus. The liquid can be aqueous, and may, for example, be water, or an aqueous liquid which is isotonic with the liquid expected to be found within the colon.

FIG. 1 shows the distal portion of the colonoscope 2 located within a colon 10, which is also shown only in part. Within the colon are solid, or semi-solid, stools, indicated diagrammatically by 11. The effect of the jets of liquid emerging from the outlets 7 is to break up the stools into a slurry and propel the slurry towards the anus, from which the slurry exits. The collection of the slurry is discussed further below.

The conduit 5 and endoscope main body of the colonoscope 2 are held adjacent one another or otherwise operatively associated with on another by suitable means. For instance, the conduit and main body may be formed integrally with one another, or they could be connected by for example guides or straps. Where the conduit and main body are not integral with one another the connection between them could be such as to permit at least limited movement of the two components with respect to one another by, for example, about 1 to about 2 cm. Such an arrangement makes possible movement of the conduit forward with respect to the main body. For instance, the conduit may be moved into the colon first by a short distance and then, when a region has been cleaned, the endoscope main body moved forward into the colon. The proximal ends of the conduit 5 and/or endoscope main body may be provided with stops to limit the extent of relative movement, and the conduit 5 may be provided with one or more markers, visible to the physician, to indicate the relative positions of the distal ends of the conduit and main body. Where stops are provided, these may be adjustable, so that the extent of relative movement can be adjusted to suit one or more specific applications. It is also to be noted that although the conduit 5 is shown mounted on the exterior of the main body, it could alternatively be passed down the biopsy channel of the endoscope or, where there is more than one biopsy channel, down one of those channels.

As the endoscope is pushed forward the wall of the colon may be encountered. In one embodiment, the closed end 6 of the conduit 5 can be formed such that if it were to strike the wall of the colon it would not cause trauma to the mucosa, and would not cause excessive discomfort to the patient. For example, the closed end 6 may carry a distal end portion, (such as in the form of a bumper) formed of a relatively soft, compliant material, such as silicone rubber. Alternatively, where the conduit for the liquid is disposed to extend through a biopsy channel, a relatively hard, rounded distal end portion can be employed, similar to the rounded end of a standard endoscopic biopsy forceps.

The supply of liquid 7 may be provided by a pump with an output provided at a constant flow rate, or alternatively, at a constant pressure. If a constant pressure is desired, the liquid may be supplied from a reservoir which provides a constant pressure head. In an alternative embodiment, rather than provide a continuous flow of liquid a pulsed flow could be used. A pulsed flow could be employed to use a relatively smaller amount of liquid in a given time period. By way of example, one second cleaning pulses with a peak flow rate of approximately 15 ml/s may be employed, with successive pulses being separated by an interval of a few seconds to allow the liquid to drain.

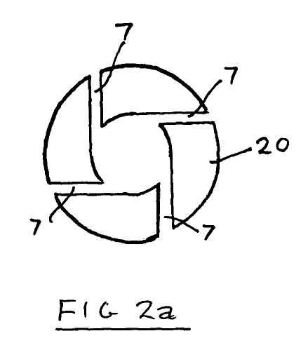

FIG. 2 illustrates an embodiment wherein the liquid outlets are formed in a separate tip member 20 mounted on the distal end of the conduit 5 in such a way as to be rotatable with respect to the remainder of the conduit. The tip member 20 may include outlets oriented to provide flow directly at least partially circumferentially. As shown in FIG. 2a, the outlets can be angled with respect to lines radial to the conduit as viewed looking at the end of the conduit in an axial direction (they may also be angled backwardly, as shown in FIG. 2), and this has the effect of causing the tip to rotate, in the same manner as a garden sprinkler used to water a lawn. The use of such a rotating tip helps to ensure that all regions of the colon are thoroughly cleaned.

The flow of liquid can be measured using pressure and flow rate indicators. It is also desirable to include in the slurry-collecting arrangement a volumetric indicator to indicate the rate at which slurry is exiting the anus, and/or the total amount of slurry which has exited. A comparison can then be made between the rate at which liquid is being supplied to the conduit 5 and the rate at which slurry is exiting, or between the total amount of liquid introduced up to a given point in time and the total amount of slurry which has exited up to that point in time. This provides a check to ensure that liquid is not accumulating in the colon.

As mentioned above, means can be provided for collecting the slurry produced as a result of the above described cleaning operation. As part of any collection arrangement it may be desirable to provide some form of fluid control arrangement at the location of the patient's anus, so as to provide a convenient way of controlling flow of the slurry into whatever receptacle may be provided to receive it. A suitable control arrangement may include a means (for example a tube) for directing the slurry to the collection receptacle, as well as an opening through which the main body and fluid conduit of the endoscope can pass. A seal can be provided around that opening to prevent leakage of the slurry. If desired, an elastic seal (such as an elastic seal ring), inflatable seal, or other suitable sealing member can be employed. The collection receptacle may, for example, be in the form of a bag. The bag may be supported by (or be otherwise associated with) a weighing device that can employed for determining the quantity of slurry exiting the anus to be compared with the quantity of liquid being introduced through it.

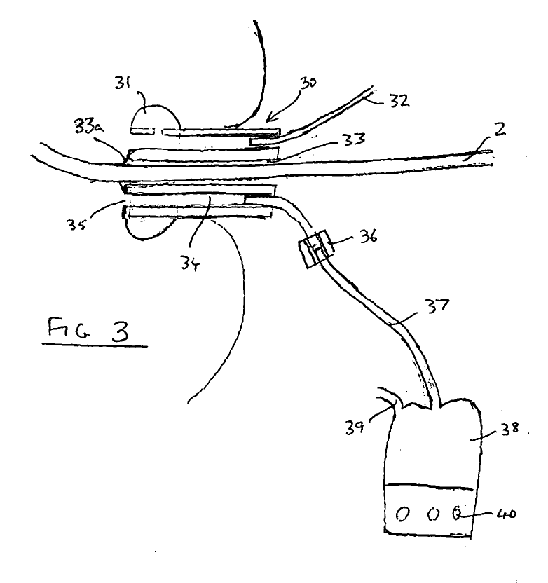

FIG. 3 shows one possible arrangement for collection of the slurry produced by the cleaning method. A bung 30 can be provided for insertion into the patient's anus. The bung can be provided on the exterior of its forward end with an inflatable balloon 31, to which inflation fluid is provided from the exterior of the patient through an inflation tube 32. The bung is inserted with the balloon in its uninflated condition, and is subsequently inflated so as to hold the bung in position. The bung can provide a channel 33 through which the colonoscope 2 can be slidably inserted into the patient's colon. The channel 33 can be provided with a seal 33a, shown here as a lip seal mounted at the distal end of the channel 33, which engages the colonoscope 2 to form a sealing connection therewith. The bung can further provide a suction channel 34 which terminates at its distal end in an aspiration port 35, and which is connected, by means of a detachable connector 36, at its proximal end to a slurry collection tube 37. This in turn is connected at its proximal end to a collection container 38, which has a port 39 for applying suction to the interior thereof. By means of this arrangement the slurry, which may include small solid pieces of faecal matter 40, is sucked into the container.

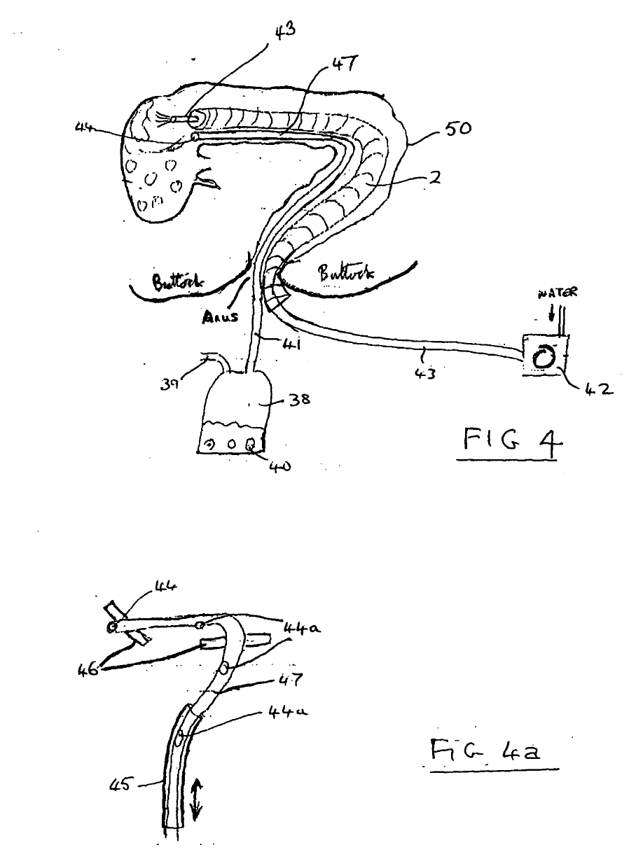

In an alternative embodiment shown in FIG. 4, the suction tube and collection container are similar to those shown in FIG. 3, but suction is applied by means of a suction tube 41 attached to the exterior of the colonoscope 2. The cleaning fluid is pumped by a pump 42 down a tube 43 inserted through the biopsy channel of the colonoscope, and emerges from the distal end of the tube 43 as a jet of water. The collection tube 47 may have a single port 44 at its distal end as shown in FIG. 4, or, as shown in FIG. 4a, it may have additional ports 44a arranged along the length thereof. In the arrangement of FIG. 4a, the collection tube 47 is received within an outer tube 45, which is longitudinally slideable with respect to the collection tube, the outer tube serving to cover such of the ports as may be located at any given time outside the patient's colon 50. This is to prevent such ports serving merely to admit air, and thus detracting from the suction of material from the interior of the colon. FIG. 4a also shows two straps 46 (there may be only a single strap, or there may be more than two) for attaching the collection tube 47 to the colonoscope 2. For this purpose the straps may, for example, be provided with adhesive.

Although the slurry collection arrangements just described have used a tube separate from the colonoscope, it is possible, provided the contents of the colon are broken up sufficiently by the jets of cleaning liquid, to aspirate the slurry through a biopsy channel of the colonoscope. If a biopsy channel is used to aspirate the slurry, the liquid for the cleaning jets may be fed through a tube exterior to the colonoscope. Alternatively, a colonoscope may be used which has more than one biopsy channel, with one channel being employed for washing and another channel being employed for suctioning. In yet another, the washing and suctioning may be done sequentially rather than simultaneously, and if desired, the same channel used for washing may be employed for suctioning.

It is also noted that although the above description has focussed on a colonoscope, the invention is also applicable to other instruments, for examining other body cavities, for example the small intestine, stomach or pancreas. The endoscope and the conduit may be introduced through any suitable opening in the body which provides access to a desired location in the body. Suitable openings, depending on the application, include surgical openings and naturally occurring orifices such as, but not necessarily limited to the mouth, nose, or anus. In the case of procedures for examining the small intestine or stomach, the endoscope would normally be introduced through the mouth, though it may be desirable in some applications to employ the devices and methods of the present invention to gain such access through the anus.

While various embodiments of the present invention have been disclosed, it will be obvious to those skilled in the art that such embodiments are provided by way of example only. Further, each element or component of the present invention may be alternatively described as a means for performing the function or functions performed by the element or component. Numerous variations, changes, and substitutions will now occur to those skilled in the art without departing from the invention. Accordingly, it is intended that the invention be limited only by the spirit and scope of the appended claims.

1. An endoscope comprising:
a main body having a distal end for insertion into a cavity in a human or animal body, and a proximal end;
viewing optics having a viewing port at or adjacent the distal end of the main body, whereby to view a region distal of the viewing port; and
a liquid conduit having a distal end portion with at least one outlet at or adjacent the distal end of the main body, and a proximal end portion arranged, in use, to receive a supply of liquid, said at least one outlet being arranged, in use, to direct said liquid to clean the cavity in the region being viewed.

2. An endoscope according to claim 1, wherein at least one outlet is located distally of the the viewing port.

3. An endoscope according to claim 1 wherein at least one outlet is arranged to direct liquid at least partially towards the proximal end of the said main body.

4. An endoscope according to claim 1 , wherein the conduit comprises an elongate tube and a tip member located at the distal end of the elongate tube and arranged to rotate with respect to the elongate tube, the or each said outlet being provided in the tip member.

5. An endoscope according to claim 1 wherein the liquid conduit is disposed exterior of the main body.

6. An endoscope according to claim 1 wherein the liquid conduit passes through a channel formed within the main body.

7. An endoscope according to claim 1 wherein the liquid conduit is movable longitudinally with respect to the main body. END5343USNP

8. An endoscope according to claim 1 in combination with a supply of liquid.

9. A combination according to claim 8 wherein the liquid supply is arranged to provide a constant volumetric flow rate.

10. A combination according to claim 8 wherein the liquid supply is a pulsed supply.

11. A combination according to claim 8, further comprising means for monitoring the liquid supply.

12. A combination according to claim 8, further comprising means for monitoring liquid leaving the cavity.

13. A combination according to claim 8, further comprising means for comparing liquid supplied with liquid leaving the cavity.

14. A combination according to claim 8, further comprising means for collecting slurry produced by the cleaning operation.

15. A combination according to claim 14, wherein the slurry collecting means comprises an aspiration tube which has at least one aspiration port and is arranged to extend into the cavity.

16. A combination according to claim 15, wherein a plurality of aspiration ports are provided, disposed lengthwise of the aspiration tube, and means are provided for covering any such port which is located at a given time outside the cavity.

17. A method for cleaning a portion of a body cavity in a patient, the method comprising:
positioning a viewing port within the cavity;
positioning a fluid conduit having at least one outlet viewable by the viewing port;
directing liquid from the outlet to clean the cavity; and
collecting at least some of the liquid used in cleaning.

18. The method of claim 17 comprising providing a substantially constant flow rate of liquid.

19. The method of claim 17 comprising providing a pulsed flow of liquid.

20. The method of claim 17 comprising monitoring the amount of liquid directed from the outlet.

21. The method of claim 17 comprising monitoring liquid leaving the cavity.

22. The method of claim 17 comprising the step of comparing the amount of liquid supplied to the cavity with the amount of liquid leaving the cavity.

23. The method of claim 17 comprising forming a slurry comprising material disposed within the cavity.

24. A method for cleaning a portion of a body cavity in a patient, the method comprising:
positioning a viewing port within the cavity;
positioning a fluid conduit having at least one outlet viewable by the viewing port;
directing liquid from the outlet; and
forming a slurry comprising material disposed within the cavity.
